(19) United States
(12) Patent Application Publication
(10) Pub. No.: US 2012/0164663 A1
(43) Pub. Date: Jun. 28, 2012

(54) Methylated Arginine Metabolites as Risk Predictors of Cardiovascular Disease

(75) Inventor: Stanley L. Hazen, Pepper Pike, OH (US)

(73) Assignee: THE CLEVELAND CLINIC FOUNDATION, Cleveland, OH (US)

(21) Appl. No.: 13/378,461

(22) PCT Filed: Jun. 15, 2010
(86) PCT No.: PCT/US10/38624

§ 371 Date: Mar. 16, 2012

Related U.S. Application Data
(60) Provisional application No. 61/187,079, filed on Jun. 15, 2009.

(51) Int. Cl. G01N 33/53; H01J 49/26; G01J 1/42; G01N 33/566

(52) U.S. Cl. 435/7.9; 436/501; 250/282; 250/395

(57) ABSTRACT

Methods for using methylated arginine metabolites and the arginine methylation index as markers for cardiovascular disease are described: The methods typically include determining the levels of dimethylarginine and N-monomethylarginine in a biological sample, comparing the levels of dimethylarginine and N-monomethylarginine to obtain an arginine methylation index; comparing the arginine methylation index to one or more control values; and using this comparison to characterize the subject's risk of having or developing cardiovascular disease or various complications associated therewith.

### CONTINUING APPLICATION DATA

This application claims the benefit of U.S. Provisional Application Ser. No. 61/187,079, filed Jun. 15, 2009, the disclosure of which is incorporated by reference herein.

### GOVERNMENT FUNDING

The present invention was made with government support by the National Institutes of Health grants P01 HL076491-055328, P01 HL087018-020001, and P50 HL077107-050004 (S.L.H.). The Government may have certain rights in this invention.

### BACKGROUND

Cardiovascular disease (CVD) accounts for one in every two deaths in the United States and is the number one killer disease. Prevention of cardiovascular disease is therefore an area of major public health importance. A low-fat diet and exercise are recommended to prevent CVD. In addition, a number of therapeutic agents may be prescribed by medical professionals to those individuals who are known to be at risk for developing or having CVD. More aggressive therapy, such as administration of multiple medications or surgical intervention may be used in those individuals who are at high risk. Since CVD therapies may have adverse side effects, it is desirable to have methods for identifying those individuals who are at risk, particularly those individuals who are at high risk, of developing or having CVD.

Currently, several risk factors are used by medical professionals to assess an individual's risk of developing or having CVD and to identify individuals at high risk. Major risk factors for cardiovascular disease include age, hypertension, family history of premature CVD, smoking, high total cholesterol, low HDL cholesterol, obesity and diabetes. The major risk factors for CVD are additive, and are typically used together by physicians in a risk prediction algorithm to target those individuals who are most likely to benefit from treatment for CVD. Use of these algorithms in combination with data on risk factors is useful for predicting risk of CVD within 10 years. However, the ability of the present algorithms to predict a higher probability of developing CVD is limited. Among those individuals with none of the current risk factors, the 10-year risk for developing CVD is still about 2%. In addition, a large number of CVD complications occur in individuals with apparently low to moderate risk profiles, as determined using currently known risk factors. Accordingly, there remains a need for methods to identify a larger spectrum of individuals who are at risk for or affected by CVD.

Atherosclerosis is known to contribute to the likelihood of developing CVD. However, the mechanisms involved in the development of atherosclerosis is not well understood. Over the past decade a wealth of clinical, pathological, biochemical and genetic data support the notion that atherosclerosis is a chronic inflammatory disorder. Acute phase reactants (e.g. C-reactive protein, complement proteins), sensitive but non-specific markers of inflammation, are enriched in fatty streaks and later stages of atherosclerotic lesions. In a recent prospective clinical trial, base-line plasma levels of C-reactive protein independently predicted risk of first-time myocardial infarction and stroke in apparently healthy individuals. U.S. Pat. No. 6,040,147 describes methods which use C-reactive protein, cytokines, and cellular adhesion molecules to characterize an individual's risk of developing a cardiovascular disorder. Although useful, these markers may be found in the blood of individuals with inflammation due to causes other than CVD, and thus, these markers may not be specific enough. Moreover, modulation of their levels has not been shown to predict a decrease in the morbidity or mortality of CVD.

Nitric oxide (NO), produced by the oxidation of arginine by NO synthases of endothelial cells, plays an important anti-atherogenic role in the development of cardiovascular disease. NO promotes many beneficial effects in the vasculature, including vasodilation, enhanced fibrinolysis and inhibition of multiple atherothrombotic biological processes including platelet aggregation, leukocyte adhesion, endothelin generation, and smooth muscle cell proliferation. Asymmetric dimethylarginine (ADMA) has been demonstrated in a variety of clinical settings to serve as an independent risk factor for long-term adverse cardiovascular events. See Boger et al., Circulation. 98, p. 1842-1847 (1998). The mechanistic explanation has been attributed to its role as an endogenous inhibitor of nitric oxide synthase (NOS), reducing the production of NO thereby diminishing vascular reactivity and leading to endothelial dysfunction and vasculopathy. Juonala et al., Circulation. 116, p. 1367-1373 (2007).

Recent identification of a family of alternative methyl-amino acid derivatives generated by related methylation pathways have provided insight into the overall protein methylation pathways that generate ADMA and other derivatives that may possess similar biological activities. Nicholls et al., Circulation, 116, p. 2315-2324 (2007). For example, N-mono-methylarginine (MMA), the immediate precursor to ADMA, is a more potent albeit less abundant endogenous NOS inhibitor. Symmetric dimethylarginine (SDMA), an alternative methylation product of MMA and a stereoisomer of ADMA, lacks NOS inhibitory activity, but like ADMA, is a weak inhibitor of arginine transporters. These derivatives are released from the methylated protein arginines by proteolysis, as shown in FIG. 1.

In contrast, methylation of other amino acids such as the production of methyl-lysine occurs via alternative methyltransferases and has no known relationship with arginine-NO metabolic pathways. The methyltransferases responsible for arginine methylation, the proteases involved in liberation of the free methylarginine derivatives and the catabolic dimethylarginine dimethylaminohydrolases involved in metabolism of ADMA are all apparently influenced by conditions associated with inflammation and oxidative stress, raising the possibility that some of the observed associations between ADMA levels and cardiovascular disease may occur in part via mechanisms independent of endogenous NOS inhibition. Pope et al., Am J Physiol Cell Physiol.; 293, C1679-1686 (2007). Arginine has previously been identified as predictive of adverse long-term cardiovascular outcomes. Nicholls et al., Circulation, 116, 2315-2324 (2007). Apart from ADMA, the relationship between other methylated arginine metabolites and prevalence of significantly obstructive coronary artery disease (CAD) and incident adverse cardiac events such as myocardial infarction (MI), stroke, and death, as well as their interactions with measures of global arginine bioavailability have not yet been explored.

### SUMMARY OF THE INVENTION

The present invention makes use of methylated arginine metabolites as diagnostic and prognostic markers for cardiovascular disease and the complications associated therewith. In one aspect, the present invention provides method of identifying a subject's risk of experiencing a complication of cardiovascular disease that includes determining the levels of dimethylarginine and N-monomethylarginine in a biological sample obtained from the subject using an analytic device; comparing the levels of dimethylarginine and N-monomethylarginine to obtain an arginine methylation index; comparing the arginine methylation index to one or more control values; and characterizing the subject's risk of experiencing a complication of cardiovascular disease as higher if the arginine methylation index is higher than the one or more control values and lower if the arginine methylation index is lower than the one or more control values.

In one embodiment, the method includes identifying a subject's risk of experiencing a complication of cardiovascular disease within the near term. In another embodiment, the complication is one or more complications selected from the group consisting of heart failure, non-fatal myocardial infarction, stroke, angina pectoris, transient ischemic attacks, aortic aneurysm, aortic dissection, peripheral artery disease, cardiomyopathy, abnormal cardiac catheterization, abnormal cardiac imaging, stent or graft revascularization, risk of experiencing an abnormal stress test, risk of experiencing abnormal myocardial perfusion, and death. In a further embodiment, the subject is experiencing chest pains and the complication is a myocardial infarction, reinfarction, acute coronary syndrome, unstable angina, or death within the near term.

Another aspect of the invention provides a method of characterizing a subject's risk of having cardiovascular disease that includes determining the levels of dimethylarginine and N-monomethylarginine in a biological sample obtained from the subject using an analytic device; comparing the levels of dimethylarginine and N-monomethylarginine to obtain an arginine methylation index; comparing the arginine methylation index to one or more control values, and characterizing the subject's risk of having cardiovascular disease as higher if the arginine methylation index is higher than the one or more control values and lower if the arginine methylation index is lower than the one or more control values.

Another aspect of the invention provides a method of characterizing a subject's risk of developing cardiovascular disease that includes determining the levels of dimethylarginine and N-monomethylarginine in a biological sample obtained from the subject using an analytic device; comparing the levels of dimethylarginine and N-monomethylarginine to obtain an arginine methylation index, comparing the arginine methylation index to one or more control values, and characterizing the subject's risk of developing cardiovascular disease as higher if the arginine methylation index is higher than the one or more control values and lower if the arginine methylation index is lower than the one or more control values.

A further aspect of the invention provides a method of evaluating the efficacy of cardiovascular therapeutic intervention in a subject with cardiovascular disease that includes determining the levels of dimethylarginine and N-monomethylarginine using an analytic device in a biological sample obtained from the subject during or after cardiovascular therapeutic intervention; comparing the levels of dimethylarginine and N-monomethylarginine to obtain an arginine methylation index; comparing the arginine methylation index to a predetermined value; and determining the cardiovascular therapeutic intervention to be efficacious if the arginine methylation index is lower than the predetermined value.

In an embodiment of this aspect of the invention, the cardiovascular therapeutic intervention is administration of a therapeutic agent, whereas in another embodiment the cardiovascular therapeutic intervention is a life style change. In a further embodiment, the predetermined value is based on the arginine methylation index derived from a comparable biological sample taken from the subject prior to cardiovascular therapeutic intervention.

In all aspects of the invention, in some embodiments the dimethylarginine is SDMA, in other embodiments the dimethylarginine is ADMA, and in other embodiments the dimethylarginine is (SDMA+ADMA). Likewise, in all aspects of the invention, in some embodiments the biological sample is blood serum, plasma, urine, or sputum. In further embodiments, the analytic device is an ultraviolet/visible detector or mass spectrometer.

### BRIEF DESCRIPTION OF THE FIGURES

The present invention may be more readily understood by reference to the following drawings wherein:

FIG. 1 provides a schematic illustration of the pathways for the production of methyl derivatives of amino acids.

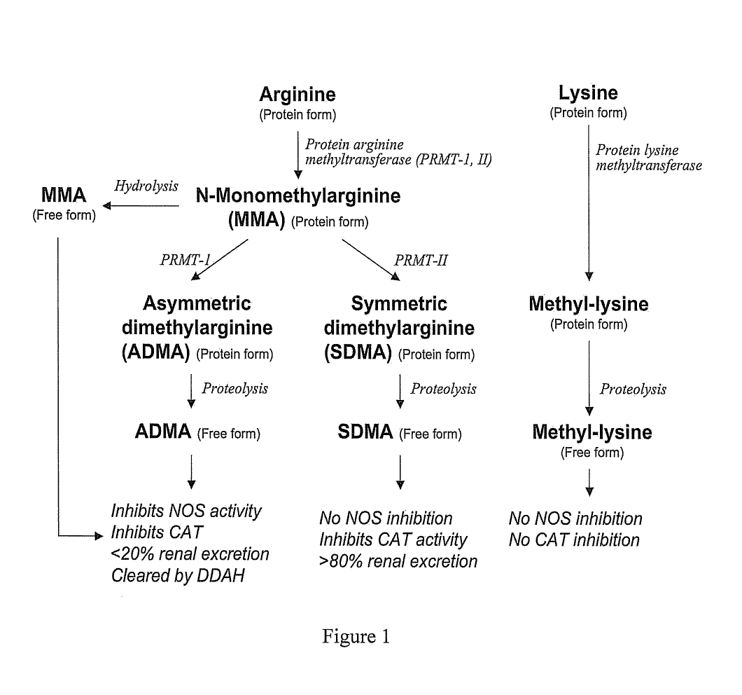

FIG. 2 provides bar graphs showing the distribution of MI/stroke, all-cause mortality, and the composite MACE according to methylated amino acid metabolites quartiles (ADMA, SDMA, MMA, and methyl-lysine).

FIG. 3 provides line graphs showing the Kaplan-Meier survival analysis for patients with major adverse cardiac events at 3-year follow-up according to the quartiles of arginine metabolites quartiles (ADMA, SDMA, MMA and arginine methylation index).

### DETAILED DESCRIPTION OF THE INVENTION

The present invention relates to the field of cardiovascular disease. More specifically, it relates to markers and methods for determining whether a subject, particularly a human subject, is at risk of developing cardiovascular disease, having cardiovascular disease, or experiencing a complication or adverse cardiac event. The present application also relates to the use of methylated arginine metabolites or the arginine methylation index for monitoring the status of cardiovascular disease in a subject or the effects of therapeutic agents on subjects with cardiovascular disease.

### General Definitions

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, peripheral vascular disease, coronary artery disease (CAD), peripheral artery disease (PAD), and cerebrovascular disease.

As used herein, the term “atherosclerotic cardiovascular disease” refers to a subset of cardiovascular disease that include atherosclerosis as a component or precursor to the particular type of cardiovascular disease. Representative examples of atherosclerotic cardiovascular disease include CAD, PAD, and cerebrovascular disease. Atherosclerosis is a chronic inflammatory response that occurs in the walls of arterial blood vessels. It involves the formation of atheromatous plaques that can lead to narrowing (“stenosis”) of the artery, and can eventually lead to partial or complete closure of the arterial opening or plaque ruptures. Thus atherosclerotic cardiovascular diseases include the consequences of atheromatous plaque formation and rupture including, without limitation, stenosis or narrowing of arteries, heart failure, aneurysm formation including aortic aneurysm, aortic dissection, and ischemic events such as myocardial infarction and stroke

A cardiovascular event, as used herein, refers to the manifestation of an adverse condition in a subject brought on by cardiovascular disease, such as sudden cardiac death or acute coronary syndromes including, but not limited to, myocardial infarction, unstable angina, aneurysm, or stroke. The term “cardiovascular event” can be used interchangeably herein with the term cardiovascular complication. Because diseases are often referred to by the complications that result therefrom, there is significant overlap in the terms used for cardiovascular disease and cardiovascular complications. While a cardiovascular event can be an acute condition (i.e., a brief and typically severe condition), it can also represent the worsening of a previously detected condition to a point where it represents a significant threat to the health of the subject, such as the enlargement of a previously known aneurysm or the increase of hypertension to life threatening levels. Examples of cardiovascular complications include heart failure, non-fatal myocardial infarction, stroke, angina pectoris, transient ischemic attacks, aortic aneurysm, aortic dissection, peripheral artery disease, cardiomyopathy, abnormal cardiac catheterization, abnormal cardiac imaging, stent or graft revascularization, risk of experiencing an abnormal stress test, risk of experiencing abnormal myocardial perfusion, and death.

### Specific Cardiovascular Condition Definitions:

Heart failure is a form of cardiovascular disease is a condition in which a problem with the structure or function of the heart impairs its ability to supply sufficient blood flow to meet the body's needs, characterized by compromised ventricular systolic or diastolic functions, or both. Heart failure may be manifested by symptoms of poor tissue perfusion alone (e.g., fatigue, poor exercise tolerance, or confusion) or by both symptoms of poor tissue perfusion and congestion of vascular beds (e.g., dyspnea, chest rates, pleural effusion, pulmonary edema, distended neck veins, congested liver, or peripheral edema). Congestive heart failure represents a form of heart failure where cardiac output is low, in contrast with high output cardiac failure, in which the body's requirements for oxygen and nutrients are increased, and demand outstrips what the heart can provide.

Heart failure can occur as a result of one or more causes. A major cause is secondary atherosclerotic disease, where one or more ischemic events such as a heart attack result in ischemic injury to the heart and decreased function. This type of heart failure is referred to as ischemic heart failure, because the cause of the cardiac dysfunction was secondary to the ischemic injury. Ischemic heart failure can also result from other cardiovascular conditions leading to ischemic injury, such as atherosclerosis that limits blood flow.

Heart failure can also occur as a result of causes other than ischemia, and such forms of heart failure are referred to as non-ischemic heart failure. Examples of non-ischemic heart failure include myocarditis resulting from viral infection, amyloidosis of cardiac tissue, arrhythmia, manifestation of genetic defects, injury from abuse of alcohol, drugs, or cigarettes, other sources of injury to cardiac tissue such as infection by bacteria or parasites, or vitamin deficiency.

Aortic dissection is a tear in the wall of the aorta that causes blood to flow between the layers of the wall of the aorta and force the layers apart. In an aortic dissection, blood penetrates the intima, which is the innermost layer of the aortic artery, and enters the media layer. The high pressure rips the tissue of the media apart along the laminated plane splitting the inner ⅔ and the outer ⅓ of the media apart. This can propagate along the length of the aorta for a variable distance forward or backwards. Dissections that propagate towards the iliac bifurcation (with the flow of blood) are called anterograde dissections and those that propagate towards the aortic root (opposite of the flow of blood) are called retrograde dissections. The initial tear is usually within 100 mm of the aortic valve so a retrograde dissection can easily compromise the pericardium leading to a hemocardium. Aortic dissection is a severe cardiovascular complication and can quickly lead to death, even with optimal treatment.

Symptoms of aortic dissection are known to those skilled in the art, and include severe pain that had a sudden onset that may be described as tearing in nature, or stabbing or sharp in character. Some individuals will report that the pain migrates as the dissection extends down the aorta. While the pain may be confused with the pain of a myocardial infarction, aortic dissection is usually not associated with the other signs that suggest myocardial infarction, including heart failure, and ECG changes. Individuals experiencing an aortic dissection usually do not present with diaphoresis (profuse sweating). Individuals with chronic dissection may not indicate the presence of pain. Aortic insufficiency is also typically seen. Other less common symptoms that may be seen in the setting of aortic dissection include congestive heart failure (7%), syncope (9%), cerebrovascular accident (3-6%), ischemic peripheral neuropathy, paraplegia, cardiac arrest, and sudden death. Preferably, this diagnosis is made by visualization of the intimal flap on a diagnostic imaging test such as a CT scan of the chest with iodinated contrast material and a trans-esophageal echocardiogram.

An aortic aneurysm, on the other hand, is a cardiovascular disorder characterized by a swelling of the aorta, which is usually caused by an underlying weakness in the wall of the aorta at that location. Aortic aneurysms are classified by where they occur on the aorta. Abdominal aortic aneurysms, hereafter referred to as AAAs, are the most common type of aortic aneurysm, and are generally asymptomatic before rupture. The most common sign for the aortic aneurysm is the Erydema Nodosum also known as leg lesions typically found near the ankle area. AAAs are attributed primarily to atherosclerosis, though other factors are involved in their formation. An AAA may remain asymptomatic indefinitely. There is a large risk of rupture once the size has reached 5 cm, though some AAAs may swell to over 15 cm in diameter before rupturing. Only 10-25% of patients survive rupture due to large pre- and post-operative mortality.

Symptoms of an aortic aneurysm may include: anxiety or feeling of stress; nausea and vomiting; clammy skin; rapid heart rate. However, an intact aortic aneurysm may not produce symptoms. As they enlarge, symptoms such as abdominal pain and back pain can develop. Compression of nerve roots may cause leg-pain or numbness. Untreated, aneurysms tend to become progressively larger, although the rate of enlargement is unpredictable for a given individual. In some cases, clotted blood which lines most aortic aneurysms can break off and result in an embolus. Preferably, medical imaging is used to confirm the diagnosis of an aortic aneurysm.

As used herein, the term “diagnosis” can encompass determining the nature of disease in a subject, as well as determining the severity and probable outcome of disease or episode of disease or prospect of recovery (prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom.

The term therapy, as used herein, encompasses both the treatment or prevention of a disease. The term “intervention” as used herein refers to the specific activity carried out to conduct therapy, and can include use of surgery, life style changes (e.g. change in diet, exercise regime, weight loss, etc.), or the use of one or more therapeutic agents targeted at CVD, (e.g. anti-inflammatory drugs, cholesterol lowering drugs, etc.).

Methylated arginine metabolites, as used herein, refers to arginine that has been methylated by metabolic processes within a subject. Methylated arginine metabolites include N-monomethylarginine (MMA) which has been methylated once, and dimethylarginines; asymmetric dimethylarginine (ADMA) and symmetric dimethylarginine (SDMA).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods or materials in connection with which the publications are cited.

As used herein and in the appended claims, the singular forms “a”, “and”, and “the” include plural referents unless the context clearly dictates otherwise. Thus, for example, reference to “a sample” includes a plurality of such samples and reference to “the dimethylarginine” includes reference to one or more dimethylarginine molecules and equivalents thereof known to those skilled in the art, and so forth.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

The present invention involves the use of methylated arginine metabolites or the arginine methylation index in relation to cardiovascular disease. In one embodiment, the invention includes determining the presence of or level of symmetric dimethylarginine (SDMA) in a bodily sample obtained from the subject. In another embodiment, the invention includes determining the presence of or level of asymmetric dimethylarginine (ADMA) in a bodily sample from the subject. In another embodiment, the invention includes determining the presence of or level of N-monomethylarginine (MMA) in a subject. In yet another embodiment, the present methods include determining the presence of or levels of dimethylarginine and N-monomethylarginine in a subject and comparing these to obtain an arginine methylation index (Arg MI) in the subject. The “arginine methylation index” maintains its prognostic value independent of measures of arginine bioavailability. Methylated arginine metabolites may be determined using a number of techniques, including: immunological techniques, mass spectrometry, high performance liquid chromatography (HPLC), or any combination thereof, as further described herein.

In one embodiment of the invention, a method of characterizing the subject's risk of having cardiovascular disease is provided. The actual presence of cardiovascular disease (CVD) can be independently confirmed using standard protocols for diagnosing CVD. Because the diagnostic markers of the present invention typically provide probabilistic information rather than conclusive “yes or no” information regarding the presence of cardiovascular disease, it is proper to refer to a method of characterizing the subjects present risk of having disease. For example, the method can characterize the subject's risk of having cardiovascular disease as being 10%, 50%, or any other number between 0% and 100%. The extent of the difference between the subject's methylated arginine metabolites or Arg MI levels and the control value is also useful for characterizing the extent of the risk, thereby determining which subjects would most likely benefit from certain therapies.

In another embodiment, a method of characterizing the subject's risk of developing cardiovascular disease is provided. The risk of developing cardiovascular disease refers to the probability that the subject will develop a cardiovascular disease that they do not currently have in the future. This risk can again range in size from any number from 0% to 100%. Subjects identified as having a high risk of developing cardiovascular disease can be selectively provided with therapeutic intervention to attempt to forestall development of the disease. In one embodiment, the test subject is an apparently healthy individual. In another embodiment, the subject is not otherwise at elevated risk of having cardiovascular disease.

In certain embodiments, the subject's risk profile for CVD is determined by combining a first risk value, which is obtained by comparing levels of methylated arginine metabolites or the arginine methylation index in a bodily sample of the subject with levels of corresponding methylated arginine metabolites or the Arg MI in a control population, with one or more additional risk values to provide a final risk value. Such additional risk values may be obtained by procedures including, but not limited to, determining the subject's blood pressure, assessing the subject's response to a stress test, determining levels of myeloperoxidase, C-reactive protein, low density lipoprotein, or cholesterol in a bodily sample from the subject, or assessing the subject's atherosclerotic plaque burden.

In another embodiment, a method of characterizing a subject's risk of experiencing a complication of CVD is provided. Essentially, the risk of experiencing a complication of cardiovascular disease represents the probability that the cardiovascular disease condition will manifest itself as an observable cardiovascular complication within the near or long term future. The subject can already exhibit other risk factors, and the method may be directed to a particular cardiovascular complication. For example, the method can be used to determine if a subject presenting with chest pain is at risk of experiencing a major adverse cardiac event, such as a myocardial infarction, reinfarction, acute coronary syndrome, unstable angina, or death within the near term.

The subject's risk of having or developing cardiovascular disease or a complication thereof may occur over a variety of different time frames. For example, the subject may have a risk of developing cardiovascular disease in the long term or the near term. As used herein, the expression “long term” refers to a risk of experiencing a major adverse cardiac event within 10 years. For example, subjects who are at long term risk may be at risk of experiencing a major adverse cardiac event within 1 years, 3 years, 5 years, or 10 years. As used herein, the expression “near term” means within one year. Thus, subjects who are at near term risk may be at risk of experiencing a major adverse cardiac event within the following day, 3 months, or 6 months.

The methods of identifying whether a subject has or will develop cardiovascular disease or a complication thereof includes determining the levels of dimethylarginine (e.g., ADMA or SDMA) and N-monomethylarginine (MMA) in a biological sample obtained from the subject, using an analytic device. In some embodiments, the levels of these compounds can be used directly, or in other embodiments the levels of dimethylarginine and N-monomethylarginine are compared to obtain an arginine methylation index. The methylated arginine metabolite levels or the arginine methylation index are then compared to one or more control values to obtain a number which can be used to characterize the subjects risk. A higher number for a comparison based on dimethylarginine (ADMA or SDMA), or the Arg MI indicates that a subject's risk of having or developing CVD or a complication thereof has increased, whereas a lower number indicates that the subject's risk of having CVD has decreased. Numbers based on MMA levels provide the inverse, namely, that lower numbers indicate an increased risk, whereas higher numbers indicate a lower risk.

Also provided herein are methods for monitoring the status of CVD in a subject over time. In one embodiment, the method comprises determining the levels of methylated arginine metabolites or the Arg MI in a biological sample taken from the subject at an initial time and in a corresponding biological sample taken from the subject at a subsequent time. For those subjects who have already experienced an acute adverse cardiovascular event such as a myocardial infarction or ischemic stroke, such methods are also useful for assessing the subject's risk of experiencing a subsequent acute adverse cardiovascular event. In such subjects, an increase in levels of SDMA, ADMA, or the Arg MI indicates that the subject is at increased risk of experiencing a subsequent adverse cardiovascular event. A decrease in levels of SDMA, ADMA, or the Arg MI in the subject over time indicates that the subject's risk of experiencing a subsequent adverse cardiovascular event has decreased. Levels of MMA can also be used in the inverse fashion, namely, increased levels indicate a lower risk, and vice versa.

Another embodiment of the invention provides a method for evaluating the efficacy of cardiovascular therapeutic intervention in a subject with cardiovascular disease. The therapeutic intervention can be any of the various types of therapeutic invention described herein, such as the use of cardiovascular agents, life style changes, and surgical intervention. The method includes determining levels of methylated arginine metabolites or an Arg MI derived therefrom in a biological sample taken from the subject prior to therapy and determining the level of the corresponding methylated arginine metabolites or Arg MI in a biological sample (e.g., an equivalent sample) taken from the subject during or following therapy. A decrease in levels of SDMA or ADMA or the Arg MI in the sample taken after or during therapy as compared to corresponding levels of SDMA or ADMA or the Arg MI in the sample taken before therapy is indicative of a positive effect of the therapy on cardiovascular disease in the treated subject.

In another embodiment, the present invention relates to kits that include reagents for assessing levels of methylated arginine metabolites or the arginine methylation index in biological samples obtained from a test subject. In certain embodiments, the kits also comprise printed materials such as instructions for practicing the present methods, or information useful for assessing a test subject's risk of CVD. Examples of such information include, but are not limited to cut-off values, sensitivities at particular cut-off values, as well as other printed material for characterizing risk based upon the outcome of the assay. In some embodiments, such kits may also comprise control reagents, e.g., ADMA, SDMA, or MMA.

As noted herein, the presence of cardiovascular disease or a complication of cardiovascular disease can be confirmed using a variety of techniques known to those skilled in the art. Medical procedures for determining whether a human subject has coronary artery disease or is at risk for experiencing a complication of coronary artery disease include, but are not limited to, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA.).

Because cardiovascular disease typically is not limited to one region of a subject's vasculature, a subject who is diagnosed as having or being at risk of having coronary artery disease is also considered at risk of developing or having other forms of CVD such as cerebrovascular disease, aortic-iliac disease, and peripheral artery disease. Subjects who are at risk of having cardiovascular disease are at risk of having an abnormal stress test or abnormal cardiac catheterization. Subjects who are at risk of having CVD are also at risk of exhibiting increased carotid intimal medial thickness and coronary calcification, characteristics that can be assessed using non-invasive imaging techniques. Subjects who are at risk of having CVD are also at risk of having an increased atherosclerotic plaque burden, a characteristic that can be examined using intravascular ultrasound.

The present invention also provides a method for monitoring over time the status of CVD in a subject who has been diagnosed as having CVD. In this context, the method is also useful for monitoring the risk for atherosclerotic progression or regression in a subject with CVD. In one embodiment, the method comprises determining the levels of methylated arginine metabolites or the Arg MI in a biological sample taken from the subject at an initial time and levels of the corresponding compounds in a biological sample (e.g., an equivalent sample) taken from the subject at a subsequent time. An increase in levels of SDMA, ADMA, or Arg MI in a biological sample taken at the subsequent time as compared to the initial time indicates that the subjects CVD has progressed or worsened. A decrease in levels of SDMA, ADMA, or Arg MI indicates that the CVD has improved or regressed.

For those subjects who has already experienced an acute adverse cardiovascular event such as a myocardial infarction or ischemic stroke, such method can also be used to assess the subject's risk of having a subsequent acute adverse cardiovascular event. An increase over time in levels of the SDMA, ADMA, or the Arg MI in the subject indicates that a subject's risk of experiencing a subsequent adverse cardiovascular event has increased. A decrease over time in levels of SDMA, ADMA, or the Arg MI in the subject indicates that that the subject's risk of experiencing a subsequent adverse cardiovascular event has decreased.

In another embodiment, the present invention provides a method for evaluating therapy in a subject suspected of having or diagnosed as having cardiovascular disease. The method comprises determining levels SDMA or ADMA or the Arg MI in a biological sample taken from the subject prior to therapy and determining levels of the corresponding compound or the Arg MI in a biological sample (e.g., an equivalent biological sample) taken from the subject during or following therapy. A decrease in the levels of SDMA or ADMA or the Arg MI in the sample taken after or during therapy as compared to levels of the corresponding SDMA or ADMA or the Arg MI in the sample taken before therapy is indicative of a positive effect of the therapy on cardiovascular disease in the treated subject.

In another embodiment, the present invention provides systems (e.g., computer systems and/or software) that is configured to receive patient data related to SDMA, ADMA, or Arg MI levels, and optionally other patient data (e.g., related to other CVD risk factors and markers) and to calculate and display a risk score. In some such embodiments, the system employs one or more algorithms to convert the biological data into a risk score. In some embodiments, the system comprises a database that associates marker levels with risk profiles, based, for example, on historic patient data, one or more control subjects, population averages, or the like. In some embodiments, the system comprises a user interface that permits a user to manage the nature of the information assessed and the manner in which the risk score is displayed. In some embodiments, the system comprises a display that displays a risk score to the user.

### Biological Samples

“Biological sample” as used herein is meant to include any biological sample from a patient (particularly a patient having, at risk of, or suspected of having CVD), where the sample is suitable for amino acid (e.g., dimethylarginine or N-monomethylarginine) content analysis. Suitable biological samples for determining dimethylarginine and N-monomethylarginine levels in a subject include but are not limited to bodily fluids such as blood-related samples (e.g., whole blood, serum, plasma, and other blood-derived samples), urine, cerebral spinal fluid, bronchoalveolar lavage, and the like. Another example of a biological sample is a tissue sample. Dimethylarginine and N-monomethylarginine levels can be assessed either quantitatively or qualitatively, usually quantitatively. The levels of the dimethylarginine and N-monomethylarginine can be determined either in vivo or ex vivo.

A biological sample may be fresh or stored (e.g. blood or blood fraction stored in a blood bank). The biological sample may be a bodily fluid expressly obtained for the assays of this invention or a bodily fluid obtained for another purpose which can be subsampled for the assays of this invention.

In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum. In another embodiment, the sample is urine.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions at physiological pH, such as phosphate, Tris, or the like, can be used.

### Subjects

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Treatment of humans is of particular interest.

The subject is any human or other animal to be tested for characterizing its risk of CVD. In certain embodiments, the subject does not otherwise have an elevated risk of an adverse cardiovascular event. Subjects having an elevated risk of an adverse cardiovascular event include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.—hereinafter “Harrison's”).

In certain embodiments the subject is apparently healthy. “Apparently healthy”, as used herein, describes a subject who does not have any signs or symptoms of CVD or has not previously been diagnosed as having any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography.

In certain embodiments, the subject is a nonsmoker. “Nonsmoker” describes an individual who, at the time of the evaluation, is not a smoker. This includes individuals who have never smoked as well as individuals who have smoked but have not smoked tobacco products within the past year. In certain embodiments, the subject is a smoker.

In some embodiments, the subject is a nonhyperlipidemic subject. “Nonhyperlipidemic” describes a subject that is a nonhypercholesterolemic or a nonhypertriglyceridemic subject. A “nonhypercholesterolemic” subject is one that does not fit the current criteria established for a hypercholesterolemic subject. A nonhypertriglyceridemic subject is one that does not fit the current criteria established for a hypertriglyceridemic subject (See, e.g., Harrison's Principles of Experimental Medicine, 15th Edition, McGraw-Hill, Inc., N.Y.—hereinafter “Harrison's”). Hypercholesterolemic subjects and hypertriglyceridemic subjects are associated with increased incidence of premature coronary heart disease. A hypercholesterolemic subject has an LDL level of >160 mg/dL, or >130 mg/dL and at least two risk factors selected from the group consisting of male gender, family history of premature coronary heart disease, cigarette smoking (more than 10 per day), hypertension, low HDL (<35 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TG) level of >250 mg/dL. Thus, a nonhyperlipidemic subject is defined as one whose cholesterol and triglyceride levels are below the limits set as described above for both the hypercholesterolemic and hypertriglyceridemic subjects.

### Methods for Measuring Levels of Dimethylarginines and N-monomethylarginine

The levels of methylated arginine metabolites such as dimethylarginines and N-monomethylarginine can be measured using any suitable analytic method, including standard methods known in the art. For example, the levels of ADMA, SDMA, or MMA in a subject can be measured using an analytic device, which is a machine including a detector capable of identifying small organic molecules such as methylated arginine metabolites. The analytic device may be a spectrometric device, such as a mass spectrometer, an ultraviolet spectrometer, or a nuclear magnetic resonance spectrometer. A spectrometer is a device that uses a spectroscopic technique to assess the concentration or amount of a given species in a medium such as a biological sample (e.g., a bodily fluid). The analytic device used to measure the levels of dimethylarginine and N-monomethylarginine can be either a portable or a stationary device. In addition to including equipment used for detecting dimethylarginine and N-monomethylarginine, the analytic device can also include additional equipment to provide physical separation of analytes prior to analysis. For example, if the analyte detector is a mass spectrometer, it may also include a high performance liquid chromatograph (HPLC) or gas chromatograph (GC) to purify the dimethylarginine and N-monomethylarginine before their detection by mass spectrometry.

As indicated herein, mass spectrometry-based methods can be used to assess levels of methylated arginine metabolites in a biological sample. Mass spectrometers include an ionizing source (e.g., electrospray ionization), an analyzer to separate the ions formed in the ionization source according to their mass-to-charge (m/z) ratios, and a detector for the charged ions. In tandem mass spectrometry, two or more analyzers are included. Such methods are standard in the art and include, for example, HPLC with on-line electrospray ionization (ESI) and tandem mass spectrometry.

The separation device and the analyte detector may be provided and referred to as a single device. For example, mass spectrometry-based methods (e.g., LC/ESI/MS/MS) may also be used to assess levels of SDMA, ADMA, or MMA in the biological sample as shown in the examples below. Such methods are standard in the art and include, for example, HPLC with on-line electrospray ionization tandem mass spectrometry. Synthetic standard tryptic digests peptides for parent (unmodified) and modified forms can be made readily with automated peptide synthesizers using commercially available modified amino acids (e.g., amino acids modified by 9-Fluorenylmethoxycarbonyl (Fmoc). The parent molecules (e.g., arginine) will have different masses than the methylated arginine metabolites (e.g., SDMA, ADMA, or MMA) because of added moieties.

Other spectrometric methods can also be used to detect dimethylarginines and N-monomethylarginine. For example, dimethylarginine and N-monomethylarginine can be measured by HPLC using a variety of detectors including, but not limited to UV or Vis (of a derivatized form), mass spectrometry, or GC/MS. Another method that can be used to identify choline-related trimethylamine-containing compounds is nuclear magnetic resonance (NMR). Examples of NMR include proton NMR and carbon-13 NMR.

Levels of methylated arginine metabolites in the biological sample can be determined using polyclonal or monoclonal antibodies that are immunoreactive with ADMA, SDMA, or MMA. For example, antibodies immunospecific for free SDMA, ADMA, or MMA-containing peptide fragments may be made and labeled using standard procedures and then employed in immunoassays to detect the presence of free SDMA or ADMA or MMA-containing peptide fragments in the sample. Suitable immunoassays include, by way of example, radioimmunoassays, both solid and liquid phase, fluorescence-linked assays, competitive immunoassays, or enzyme-linked immunosorbent assays. In certain embodiments, the immunoassays are also used to quantify the SDMA, ADMA, and MMA that is present in the sample. Polyclonal or monoclonal antibodies raised against methylated arginine metabolites are produced according to established procedures. Generally, as an initial step, a methylated arginine-containing peptide fragment is used to immunize a host animal. These procedures are well known to those skilled in the art.

Various immunoassays may be used for screening to identify antibodies having the desired specificity. These include protocols that involve competitive binding or immunoradiometric assays and typically involve the measurement of complex formation between the methylated arginine metabolite containing fragment and the antibody. Accordingly, embodiments of the present invention provide antibodies that are immunospecific methylated arginine metabolites. Such antibodies are useful for determining or measuring the levels of one or more methylated arginine metabolites present in biological samples obtained from the subject.

The present antibodies may be used to detect the presence of or measure the amount of methylated arginine metabolites in a biological sample from the subject. The method comprises contacting a sample taken from the individual with one or more of the present antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be a tissue or a biological fluid, including urine, whole blood, or exudate, preferably serum. The sample may be untreated, subjected to precipitation, fractionation, separation, or purification before combining with the antibody. Interactions between antibodies in the sample and free methylated arginine or a methylated arginine-containing peptide or polypeptide are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of dimethylarginine and N-monomethylarginine in the subject's biological sample.

Suitable methods for determining a level of arginine compounds have been reported in the literature. See, e.g., U.S. Pat. No. 6,720,188; Teerlink et al. (2002) Anal. Biochem. 303:131-137; Dobashi et al. (2002) Analyst 127:54-59; Pi et al. (2000) J. Chromatogr. B. Biomed. Sci. Appl. 742:199-203; Chen et al. (1997) J. Chromatogr. B. Biomed. Sci. Appl. 692:467-471; Anderstam et al. (1997) J. Am. Soc. Nephrol. 8:1487-1442; Pettersson et al. (1997) J. Chromatogr. B. Biomed. Sci. Appl. 692:257-262; Sultana et al. (2001) J. Chromatogr. B. Biomed. Sci. Appln. 755:321; Chace et al. (2003) Clin. Chem. 49:1797-1817; and Trapp et al. (2004) J. Sep. Sci. 27:1483-1490.

Once the levels of dimethylarginine and N-monomethylarginine have been deter mined, they can be displayed in a variety of ways. For example, the levels of dimethylarginine and N-monomethylarginine can be displayed graphically on a display as numeric values or proportional bars (i.e., a bar graph) or any other display method known to those skilled in the art. The graphic display can provide a visual representation of the amount of the methylated arginine metabolites (e.g., ADMA, SDMA, or MMA) in the biological sample being evaluated. In addition, in some embodiments, the analytic device can also be configured to display the Arg-MI or a comparison of the Arg MI to a control value based on levels of dimethylarginine and N-monomethylarginine in comparable bodily fluids from a reference cohort.

### Calculation of the Arginine Methylation Index and Control Values

After the levels of dimethylarginine (e.g., ADMA or SDMA) and N-monomethylarginine have been obtained, these amounts may be used to calculate the arginine methylation index (Arg MI). As further described herein, the Arg MI index has been shown to be a particularly useful indicator for the risk of having or developing cardiovascular disease. Accordingly, some embodiments of the invention are directed specifically to the use of the Arg MI ratio for characterizing a subjects risk of having or developing cardiovascular disease, and other uses of methylated arginine metabolites described herein. The Arg MI represents the ratio of dimethylarginine to N-monomethylarginine. The Arg MI can be calculated using the level of a single dimethylarginine (e.g., ADMA or SDMA) compared to the level of N-monomethylarginine, or the Arg MI can be calculated using the level of the combined dimethylarginines (.e.g., ADMA and SDMA) compared to the level of N-monomethylarginine. Accordingly, the Arg MI can represent ADMA/MMA, SDMA/MMA, or (ADMA+SDMA)/MMA.

In various embodiments of the invention, the levels of methylated arginine metabolites can be compared directly to control values, or the levels of dimethylarginine can be compared to the level of MMA to obtain an Arg MI which is compared to a control value. A control value is determined by comparison to a reference cohort. The control value is based upon levels of SDMA, ADMA, MMA, or the Arg MI in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. In certain embodiments, the reference cohort is comprised of individuals, who if examined by a medical professional would be characterized as free of symptoms of disease. In another example, the reference cohort may be individuals who are nonsmokers. A nonsmoker cohort may have a different normal range of methylated arginine metabolites or Arg MI than will a smoking population or the general population. Accordingly, the control values selected may take into account the category into which the test subject falls. Appropriate categories can be selected with no more than routine experimentation by those of ordinary skill in the art.

The control value is provided in a manner that corresponds or relates to the value used to characterize the level of methylated arginine metabolites obtained from the test subject. Thus, if the level of the SDMA, ADMA, or MMA is an absolute value such as the units of SDMA, ADMA, or MMA per ml of blood, the control value is also based upon the units of SDMA, ADMA, or MMA per ml of blood in individuals in the general population or a select population of human subjects.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. Control values of methylated arginine metabolites or the Arg MI in biological samples obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each risk predictor that is assayed. A standardized method that may be used employs the guaiacol oxidation assay as described by Klebanoff et al., Methods in Enzymology. 105: 399-403 (1984).

In some embodiments of the invention, a predetermined value is used. For example, one embodiment of the invention provides a method of evaluating the efficacy of cardiovascular therapeutic intervention in a subject with cardiovascular disease that includes determining the levels of dimethylarginine and N-monomethylarginine using an analytic device in a biological sample obtained from the subject during or after cardiovascular therapeutic intervention; comparing the levels of dimethylarginine and N-monomethylarginine to obtain an arginine methylation index; comparing the arginine methylation index to a predetermined value; and determining the cardiovascular therapeutic intervention to be efficacious if the arginine methylation index is lower than the predetermined value. A predetermined value can be based on the levels of methylated arginine metabolites or the Arg MI in a biological sample taken from a subject prior to cardiovascular therapeutic intervention, such as administration of a cardiovascular therapeutic agent. In another embodiment, the predetermined value is based on the levels of methylated arginine metabolites or the Arg MI in biological samples taken from control subjects that are apparently healthy, as defined herein. A predetermined value can include levels present in subjects having been diagnoses as having cardiovascular disease. Unlike control values, predetermined values can be fairly arbitrary and need not be based on sampling of a population of subjects.

Comparison of Methylated Arginine Metabolites or the Arg MI from a Test Subject to the Control Value

Levels of methylated arginine metabolites or the Arg MI in the individual's biological sample may be compared to a single control value or to a range of control values. If the level of the present risk predictor in the test subject's biological sample is greater than the control value or exceeds or is in the upper range of control values, the test subject is at greater risk of developing or having CVD than individuals with levels comparable to or below the control value or in the lower range of control values. In contrast, if levels of the present risk predictor in the test subject's biological sample is below the control value or is in the lower range of control values, the test subject is at a lower risk of developing or having CVD than individuals whose levels are comparable to or above the control value or exceeding or in the upper range of control values. The extent of the difference between the test subject's risk predictor levels and control value is also useful for characterizing the extent of the risk and thereby determining which individuals would most greatly benefit from certain aggressive therapies. In those cases where the control value ranges are divided into a plurality of groups, such as the control value ranges for individuals at high risk, average risk, and low risk, the comparison involves determining into which group the test subject's level of the relevant risk predictor falls. Alternatively, the level of methylated arginine metabolites may be compared to the level of an internal standard in the sample.

### Evaluation of CVD Therapeutic Agents

The present predictive tests are useful for determining if and when therapeutic agents that are targeted at preventing CVD or for slowing the progression of CVD should and should not be prescribed for a individual. For example, individuals with SDMA, ADMA, or Arg MI values above a certain cutoff value, or that are in the higher tertile or quartile of a “normal range,” could be identified as those in need of more aggressive intervention with lipid lowering agents, life style changes, etc.

Also provided are methods for evaluating the effect of CVD therapeutic agents on individuals who have been diagnosed as having or as being at risk of developing CVD. Such therapeutic agents include, but are not limited to, anti-inflammatory agents, insulin sensitizing agents, antihypertensive agents, anti-thrombotic agents, anti-platelet agents, fibrinolytic agents, lipid reducing agents, direct thrombin inhibitors, ACAT inhibitor, CDTP inhibitor thioglitazone, glycoprotein II b/IIIa receptor inhibitors, agents directed at raising or altering HDL metabolism such as apoA-I milano or CETP inhibitors (e.g., torcetrapib), or agents designed to act as artificial HDL. Such evaluation comprises determining the levels of SDMA, ADMA, or Arg MI in a biological sample taken from the subject prior to administration of the therapeutic agent and a corresponding biological fluid taken from the subject following administration of the therapeutic agent. A decrease in the level of the selected risk markers in the sample taken after administration of the therapeutic as compared to the level of the selected risk markers in the sample taken before administration of the therapeutic agent is indicative of a positive effect of the therapeutic agent on cardiovascular disease in the treated subject.

### Therapeutic Methods

The present invention also relates to methods of treating a subject to reduce the risk of a cardiovascular disorder or complication of such disorder. In one embodiment, the method comprises determining the level of SDMA, ADMA or the Arg MI in a bodily sample of the subject, and where the levels of the SDMA, ADMA or the Arg MI are elevated as compared to levels in comparable bodily samples from a control population of subjects, administering to the subject an agent chosen from an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, a cyclooxygenase-2 (COX-2) inhibitor, an angiotensin system inhibitor, or combinations thereof. The agent is administered in an amount effective to lower the risk of the subject developing a future cardiovascular disorder. A wide variety of cardiovascular agents together with their recommended dosages, pharmacology, and contraindications can be found in the most recent version of the Physician's Desk Reference (currently the 59th edition), which is incorporated herein by reference. In some embodiments, levels of SDMA, ADMA or the Arg MI are assessed at one or more time points following therapy to monitor the effectiveness of the therapy and, as desired, to alter the therapy accordingly (e.g., continue therapy, discontinue therapy, change therapy).

An example has been included to more clearly describe a particular embodiment of the invention and its associated cost and operational advantages. However, there are a wide variety of other embodiments within the scope of the present invention, which should not be limited to the particular example provided herein.

### EXAMPLES

### Example 1

### Targeted Metabolomic Evaluation of Arginine Methylation and Cardiovascular Risks: Potential Mechanisms Beyond Nitric Oxide Synthase Inhibition

By simultaneous measurements of multiple amino acid methylation derivatives, the prognostic value of ADMA relative to SDMA, MMA, as well as the unrelated methylated amino acid methyl-lysine (Methyl-Lys) was evaluated in patients undergoing evaluation for coronary artery disease.

### Methods

Study Design. All plasma specimens were obtained from subjects who were prospectively enrolled in GeneBank, a large (n=10,000) and well-characterized clinical repository with clinical and longitudinal data comprised from consenting subjects undergoing elective diagnostic cardiac catheterization at the Cleveland Clinic. All GeneBank participants gave written informed consent and the Institutional Review Board of the Cleveland Clinic approved the study protocol. For the present study, 1,011 consecutive consented patients were evaluated, with clinical and demographic information collected at the time of catheterization and for whom long-term (3 year) incident outcome data were available. The Framingham Risk Score was calculated for each subject. An estimate of creatinine clearance (CrCl) was calculated using the Cockcroft-Gault equation. Cockcroft et al., Nephron, 16, 31-41 (1976) High sensitivity C-reactive protein (hsCRP), creatinine, and fasting blood glucose and lipid profiles were measured on the Abbott ARCHITECT platform (Abbott Diagnostics, Abbott Park Ill.). Adjudicated outcomes were ascertained over the ensuing 3 years for all subjects following enrollment.

Endpoint Definitions. Significantly obstructive CAD was defined as any clinical history of myocardial infarction (MI), percutaneous coronary intervention, coronary artery bypass surgery, or angiographic evidence of CAD (≧50% stenosis) in one or more major coronary arteries. Major adverse cardiovascular event (MACE) was defined as death, non-fatal myocardial infarction, or non-fatal cerebrovascular accident.

Measurements of Amino Acids. Plasma analyzed was isolated from fasting whole blood collected in EDTA tubes that had been maintained at 4° C. immediately following phlebotomy, processed typically within 2 hours (4 hours max) of blood draw, and stored at −80° C. until use. Arginine and the arginine metabolites ornithine, citrulline, MMA, ADMA and SDMA, were quantified in plasma by stable-isotope-dilution HPLC with online tandem mass spectrometry. Briefly, [¹³C₆] arginine (10 μM final) was initially added to plasma as internal standard and proteins then precipitated by addition of 4 volumes of methanol. Supernatant (20 μl) was injected onto a Phenyl column (4.6×250 mm, 5 μm Rexchrom Phenyl) (Regis, Morton Grove, Ill.) at a flow rate of 0.8 ml/min. Separation was performed using a gradient starting from 10 mM ammonium formate aqueous solution over 0.5 min, then linearly to 25% methanol containing 0.1% formic acid and 5 mM ammonium formate over 3 min, followed by isocratic run with the same solvent composition for 15 min. The HPLC column effluent was introduced into an API 365 triple quadrupole mass spectrometer with Ionics EP 10⁺ upgrade (Concord, Ontario, CA) interfaced to a Cohesive Technologies Aria LX Series HPLC multiplexing system (Franklin, Mass.). Analyses were performed using electrospray ionization in positive-ion mode with multiple reactions monitoring of parent and characteristic daughter ions specific for components monitored. The transitions monitored were mass-to-charge ratio (m/z): m/z 133.2→70.2 for ornithine; m/z 175.1→70.0 for arginine; m/z 176.1→70.1 for citrulline; m/z 189.3→470.1 for MMA; m/z 203.2→70.3 for SDMA and ADMA; and m/z 181→474 for [¹³C₆]Arg. The calibration curves for quantification of ornithine, arginine, citrulline, MMA, ADMA and SDMA were prepared by spiking different concentrations of each individual analyte to control plasma. All analytes were baseline resolved and showed unique retention times. A S/N of 3 was used as minimal for limit of detection. For ornithine, arginine, citrulline, MMA, SDMA and ADMA, the inter-assay CVs (%) were 12.1, 4.4, 6.3, 6.7, 6.5, 5.5 and the intra-assay CVs (%) were 4.8, 3.3, 8.6, 8.3, 5.5, 6.7, respectively. Assay performance characteristics included average spike and recovery of 94% (range from 84 to 110%) for all analytes monitored in plasma matrix, and assay precision of <10% across all concentration ranges monitored for all analytes under the assay conditions employed.

Separate analyses examined the methylation index for arginine (ArgMI), an integrated quantification of products generated from the arginine methylation pathways, and is estimated by the ratio of the known di-methylated arginine post translational modifications (ADMA+SDMA) to the immediate mono-methylated precursor, MMA [i.e. ArgMI=(ADMA+SDMA)/MMA]. Global arginine bioavailability ratio (GABR) has previously been defined as the ratio of substrates (arginine) and catabolic products (ornithine and citrulline) of arginine metabolism.

Statistical Analysis. Summary data were presented in mean±standard deviation for parametric variables or median (interquartile ranges) for non-parametric variables. The Student's t-test or Wilcoxon Rank sum test for continuous variables and chi-square test for categorical variables were used to examine the difference between the groups. Levels of MMA, SDMA, ADMA, methyl-Lys, and ArgMI were divided into quartiles for analyses. Unadjusted trends for all-cause mortality rates as well as non-fatal MI/stroke rates with increasing quartiles of methylated derivatives were evaluated with the Cochran-Armitage trend test. Logistic regression models were developed to calculate odds ratios (ORs) and 95% confidence intervals (95% CI) of the prevalence of coronary artery disease for the second, third and the highest quartiles of analytes (or ArgMI) compared with the lowest quartile. Adjustments were made for individual traditional cardiac risk factor or Framingham Risk Score, log-transformed hsCRP, and CrCl to predict incident 3-year MACE risks. Kaplan-Meier analysis with Cox proportional hazards regression was used for time-to-event analysis to determine Hazard ratio (HR) and 95% confidence intervals (95% CI) for MACE. Levels of analytes were then adjusted for traditional CAD risk factors in a multivariable model including Framingham Risk Score, CrCl, and log-transformed hsCRP, as well as incorporating GABR into the model.

To determine whether ArgMI has an additional predictive value compared to a model including Framingham Risk Score, we calculated the concordance indexes (C statistic) with and without ArgMI in separate multivariable models. The improvement in predictability was assessed by calculating the difference in the C statistics. Bootstrapping was used to generate 95% CI. A one-sample t-test was performed to determine if the difference was equal to zero. We defined primary and secondary prevention cohorts as those with and without the following characteristics, respectively: history of significantly obstructive CAD by coronary angiography, history of coronary revascularization (surgical or percutaneous), or history of MI or stroke.

All analyses were performed using SAS version 8.2 (Cary, N.C.). P values <0.05 were considered statistically significant.

### Results

Study Population. Table 1 illustrates the baseline characteristics of the study population, which included 608 subjects with significantly obstructive CAD classified according to our endpoints criteria at time of enrollment. As expected, subjects with significantly obstructive CAD were older, more likely to have diabetes and hypertension, and have lower CrCl, though well within the normal range. Patients with significantly obstructive CAD had higher levels of hsCRP, triglyceride, and lower levels of (HDL) cholesterol compared to those without significantly obstructive CAD (Table 1). Data presented in percent for dichotomous variables, mean±standard deviation for parametric continuous variables or median (interquartile ranges) for non-parametric continuous variables.

TABLE 1
Demographics of patients with and without significantly
obstructive coronary artery disease (CAD)

| | | |
|---|---|---|
| No Significantly | Significantly | |
| Obstructive | Obstructive | |
| CAD | CAD | |
| (n = 402) | (n = 608) | p-value |

Demographics and Cardiovascular Risk Factors

| | | | |
|---|---|---|---|
| Age (years) | 61.4 ± 7.8 | 65.6 ± 9.8 | <0.001 |
| Female (%) | 53.2% | 52.3% | 0.772 |
| Diabetes mellitus (%) | 14.5% | 45.2% | <0.001 |
| Hypertension (%) | 54.2% | 78.0% | <0.001 |
| Framingham Risk Score | 6.6 ± 3.4 | 8.5 ± 4.0 | <0.001 |
| Former smokers (%) | 50.8% | 57.2% | 0.043 |
| Current smokers (%) | 4.5% | 5.9% | 0.318 |

Laboratory Data

| | | | |
|---|---|---|---|
| LDL | 108 ± 33 | 100 ± 35 | <0.001 |
| HDL | 53 ± 17 | 44 ± 14 | <0.001 |
| Triglycerides | 117 (83, 168) | 141 (105, 212) | <0.001 |
| hsCRP, mg/dL | 2.2 (1.1, 4.6) | 3.3 (1.7, 7.6) | <0.001 |
| Creatinine clearance, | 99 ± 33 | 88 ± 40 | <0.001 |
| mL/min | | | |

Median (IQR) Arginine methylation products

| | | | |
|---|---|---|---|
| ADMA, μM | 0.97 (0.75, 1.35) | 1.09 (0.81, 1.54) | 0.003 |
| SDMA, μM | 0.59 (0.45, 0.83) | 0.71 (0.50, 1.14) | <0.001 |
| MMA, μM | 0.07 (0.05, 0.09) | 0.06 (0.05, 0.08) | 0.002 |
| Methyl-lysine, μM | 2.17 (1.37, 5.74) | 2.21 (1.36, 5.22) | 0.452 |
| ArgMI | 22.5 (18.1, 31.1) | 28.6 (20.4, 47.1) | <0.001 |

Methylation derivatives of amino acids and prevalence of coronary artery disease. Patients with significantly obstructive CAD had significantly higher median plasma levels of ADMA (1.09 μM vs 0.97 μM, p=0.003), SDMA (0.71 μM vs 0.59 μM, p<0.001), and lower levels of MMA (0.06 μM vs 0.07 μM, p=0.002) compared to those without significantly obstructive CAD (Table 1). In contrast, there was no difference between the two groups in the level of methyl-lysine (2.21 μM vs 2.17 μM, p=0.45).

For further analysis, ADMA, SDMA, MMA, and methyl-lysine were divided into quartiles. Unadjusted odds ratio for increasing ADMA and SDMA quartiles were associated with significantly obstructive CAD (Table 2). In contrast, unadjusted odds ratios for increasing MMA quartiles were paradoxically associated with lower prevalence of CAD compared to the lowest quartile (Table 2). In view of the opposite trends of association among products (positive for ADMA, SDMA) and precursors (negative for MMA) of arginine methylation with prevalence of significantly obstructive CAD, the relationship between increasing quartiles of the Arginine Methylation Index [ArgMI, defined as (ADMA+SDMA)/MMA] and significantly obstructive CAD in the cohort was further examined. Interestingly, among the methylated arginine parameters examined (i.e. ADMA, SDMA, MMA and ArgMI), the integrated methylation index, ArgMI, remained the most robust independent predictor of significantly obstructive CAD status after adjustments for traditional risk factors, renal function, and hsCRP, demonstrating odds ratios of [adjusted OR (95% CI): 1.63 (1.09-2.46), p=0.019] for the third quartile and [adjusted OR (95% CI): 2.64 (1.71-4.08), p<0.001] for fourth quartile (Table 2). Interestingly, while SDMA remained a significant predictor of significantly obstructive CAD status following adjustments [adjusted OR (95% CI): 1.64 (1.06-2.56), ADMA no longer remained significantly associated with CAD status [adjusted OR(95% CI): 1.33 (0.88-2.03)] (Table 2).

TABLE 2
Odds ratio for prevalence of significantly obstructive CAD and Hazard Ratio
for Major according to ADMA, SDMA, MMA, and methyl-lysine quartiles.

| | Odds Ratio for Prevalence of Significantly Obstructive CAD | | | | Hazard Ratio for Major Adverse Cardiac Events at 3 Years | | | |
|---|---|---|---|---|---|---|---|---|
| | 1^{st} quartile | 2^{nd} quartile | 3^{rd} quartile | 4^{th} quartile | 1^{st} quartile | 2^{nd} quartile | 3^{rd} quartile | 4^{th} quartile |
| ADMA (range, μM) | 0.087-0.78 | 0.784-1.03 | 1.04-1.48 | 1.49-8.06 | 0.087-0.78 | 0.784-1.03 | 1.04-1.48 | 1.49-8.06 |
| Unadjusted | 1.0 | 0.98 | 1.57 | 1.62 | 1.0 | 1.58 | 2.75 | 3.20 |
| (95% CI) | | (0.69, 1.39) | (1.09, 2.26)* | (1.12, 2.34)* | | (0.83, 3.00) | (1.52, 4.96)# | (1.78, 5.73)† |
| Adjusted | 1.0 | 0.89 | 1.56 | 1.33 | 1.0 | 1.60 | 1.82 | 2.16 |
| (95% CI) | | (0.60, 1.32) | (1.02, 2.37)* | (0.88, 2.03) | | (0.83, 3.08) | (0.97, 3.42) | (1.16, 4.04)* |
| SDMA (range, μM) | 0.063-0.469 | 0.47-0.654 | 0.657-1.03 | 1.05-6.17 | 0.063-0.469 | 0.47-0.654 | 0.657-1.03 | 1.05-6.17 |
| Unadjusted | 1.0 | 1.05 | 1.57 | 2.20 | 1.0 | 1.21 | 2.71 | 3.82 |
| (95% CI) | | (0.74, 1.49) | (1.10, 2.25)* | (1.52, 3.20)* | | (0.62, 2.39) | (1.50, 4.90)# | (2.15, 6.79)† |
| Adjusted | 1.0 | 0.98 | 1.61 | 1.64 | 1.0 | 1.10 | 2.08 | 2.38 |
| (95% CI) | | (0.66, 1.46) | (1.06, 2.47)* | (1.06, 2.56)* | | (0.55, 2.24) | (1.10, 3.93)* | (1.24, 4.56)# |
| MMA (range, μM) | 0.0-0.048 | 0.048-0.063 | 0.064-0.083 | 0.083-0.434 | 0.0-0.048 | 0.048-0.063 | 0.064-0.083 | 0.083-0.434 |
| Unadjusted | 1.0 | 0.80 | 0.59 | 0.54 | 1.0 | 0.56 | 1.00 | 0.89 |
| (95% CI) | | (0.55, 1.16) | (0.41, 0.85)* | (0.37, 0.78)* | | (0.33, 0.95)* | (0.63, 1.58) | (0.55, 1.44) |
| Adjusted | 1.0 | 0.57 | 0.46 | 0.55 | 1.0 | 0.59 | 0.93 | 0.87 |
| (95% CI) | | (0.38, 0.88)* | (0.30, 0.71)† | (0.35, 0.84)* | | (0.34, 1.02) | (0.58, 1.50) | (0.53, 1.43) |
| Methyl-Lys (range, μM) | 0.479-1.355 | 1.355-2.173 | 2.182-5.337 | 5.339-16.362 | 0.479-1.355 | 1.355-2.173 | 2.182-5.337 | 5.339-16.362 |
| Unadjusted | 1.0 | 0.92 | 1.17 | 0.89 | 1.0 | 1.19 | 1.28 | 0.80 |
| (95% CI) | | (0.64, 1.31) | (0.81, 1.79) | (0.62, 1.27) | | (0.73, 1.94) | (0.79, 2.07) | (0.47, 1.37) |
| Adjusted | 1.0 | 0.81 | 1.13 | 0.76 | 1.0 | 1.28 | 1.17 | 0.72 |
| (95% CI) | | (0.54, 1.22) | (0.73, 1.70) | (0.50, 1.14) | | (0.77, 2.13) | (0.70, 1.96) | (0.41, 1.28) |
| ArgMI (range) | 5.4-19.4 | 19.5-25.6 | 25.7-41.4 | 41.5-252.9 | 5.4-19.4 | 19.5-25.6 | 25.7-41.4 | 41.5-252.9 |
| Unadjusted | 1.0 | 1.10 | 1.87 | 3.42 | 1.0 | 1.64 | 3.49 | 4.15 |
| (95% CI) | | (0.77, 1.57) | (1.31, 2.69)* | (2.32, 5.05)† | | (0.82, 3.27) | (1.87, 6.50)† | (2.26, 7.65)† |
| Adjusted | 1.0 | 1.07 | 1.63 | 2.64 | 1.0 | 1.31 | 2.50 | 2.40 |
| (95% CI) | | (0.72, 1.58) | (1.09, 2.46)* | (1.71, 4.08)† | | (0.65, 2.65) | (1.33, 4.69)# | (1.28, 4.51)# |

Methylation derivatives of amino acids and incident cardiovascular risks. Among the 1,011 subjects, there were 56 patients lost to follow up. Of the remaining 955 patients, 824 patients had no MACE (non-fatal MI, stroke or death) over the 3 years following GeneBank enrollment, while 131 patients experienced at least one MACE. Patients who experienced MACE were older and more likely to have a history of diabetes and hypertension. Patients with risk for MACE also had increased CRP level, higher triglyceride, lower HDL, lower CrCl and higher Framingham Risk Score. Plasma levels of ADMA (1.27 (0.95, 1.69) vs 0.97 (0.76, 1.40) μM, p<0.001), SDMA (0.89 (0.60, 1.35) vs 0.63 (0.46, 0.93) μM, p<0.001) and ArgMI (33.3 (25.3, 53.9) vs 24.4 (19.0, 40.0), p<0.001) were significantly higher in patients who experienced a MACE over the ensuing 3 years following enrollment compared with those who did not experience a MACE. In contrast, the alternative methylated amino acid methyl-lysine (2.11 (1.40, 4.76) vs 2.18 (1.36, 5.56), p=0.37) and MMA (0.07 (0.05, 0.08) vs 0.06 (0.05, 0.08), p=0.95) levels were not significantly different between the two groups.

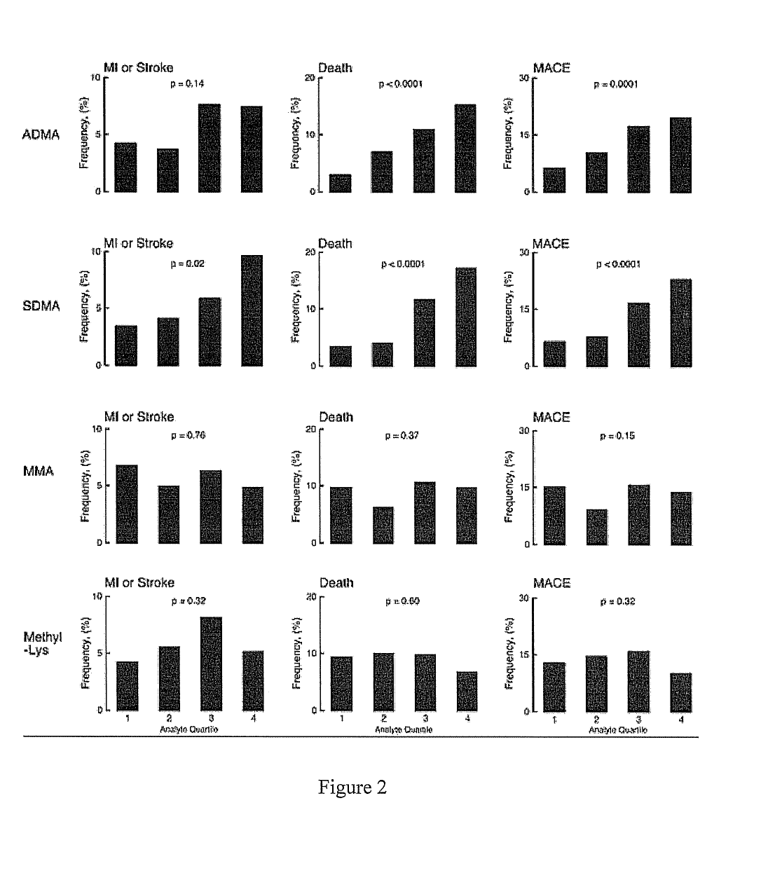

The analytes were divided into quartiles for further analyses of risk prediction, and the rates of non-fatal MI or stroke, all-cause mortality, as well as the composite MACE, are presented in FIG. 2. Interestingly, while a trend between increasing quartiles of ADMA levels and incident rates of MI or stroke failed to reach significance, systemic levels of SDMA were significantly associated with incident MI or stroke frequency (FIG. 2). In particular, there was statistically significant increased unadjusted risk for all-cause mortality at 3 years across increasing quartiles of SDMA [HR (95% CI): 5.3 (2.5-11.3)] and ADMA [HR (95% CI): 5.3 (2.3-11.8)]). In contrast, there was no association between quartiles with all-cause mortality or MACE for either MMA (p-trend=0.88) or methyl-lysine (p-trend=0.75). Also with each increasing quartile of SDMA [HR (95% CI): 1.63 (1.4-1.9)] and ADMA [HR (95% CI): 1.46 (1.2-1.7)], there was statistically significant increased unadjusted risk for future MACE.

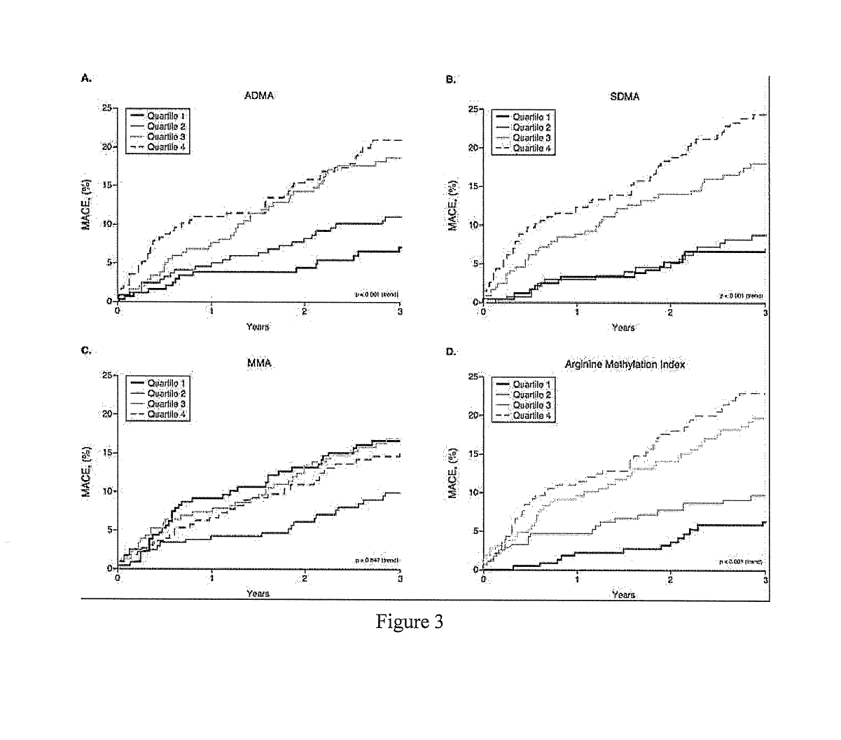

Kaplan Meier survival curves for each analyte and prospective MACE are shown in FIG. 3. Dose-dependent increases in systemic levels of both ADMA and SDMA within subjects were strongly associated with increased risk for experiencing a MACE over the 3 year monitoring period following enrollment (Table 2). After adjusting for Framingham Risk Score, hsCRP, and renal function (CrCl), subjects with higher levels of ArgMI [third quartile HR (95% CI): 2.5(1.3-4.7, p=0.005; fourth quartile HR (95% CI): 2.4(1.3-4.5), p=0.006; all compared to the first quartile] showed the greatest risk for experiencing an incident MACE over the ensuing 3 year period. Moreover, C-statistic analyses demonstrated significant incremental prognostic value in the highest ArgMI quartile plus risk factors for prediction of incident 3-year MACE risk compared to that of risk factors alone (AUC 0.78 vs 0.55, p<0.001).

Several recent studies have reported that the Global Arginine Bioavailability ratio [GABR, defined as (Ornithine+Citrulline)/Arginine] shows prognostic value for prediction of incident risk for major adverse cardiac events Morris et al., JAMA, 294, 81-90 (2005). Tang et al., J Am Coll Cardiol. 53, 2061-2067 (2009). To gain a better appreciation of the relationship between the targeted arginine metabolome and cardiovascular disease within the same cohort, the prognostic value of various arginine methylation products was examined as comprehensively monitored in the arginine methylation index (ArgMI) and the GABR. ArgMI and GABR each retained independent prognostic value in multilogistic regression models simultaneously incorporating ArgMI, GABR and other traditional cardiovascular risk factors (Table 3). Remarkably, when further stratified according to primary versus secondary prevention cohorts, ArgMI was found to be highly predictive of incident 3-year MACE risk particularly in the secondary prevention cohort, with a trend towards predictive of incident 3-year MACE risk in the primary prevention cohort (Table 3). In contrast, GABR showed a stronger predictive value among primary prevention subjects compared with secondary prevention subjects (Table 3).

TABLE 3
Cox proportional hazard analysis for 3-year major adverse clinical
events for arginine methylation and global arginine bioavailability,
adjusted for renal function and cardiac risk factors as well as stratified
according to primary and secondary prevention cohorts.

| | Overall Cohort | | Primary Prevention | | Secondary Prevention | |
|---|---|---|---|---|---|---|
| | Hazard | p | Hazard | p | Hazard | p |
| | Ratio*† | value | Ratio* | value | Ratio* | value |
| ArgMI | 2.40 | <0.01 | 4.11 | 0.068 | 3.14 | 0.001 |
| | (1.28, 4.5) | | (0.90, 18.7) | | (1.58, 6.30) | |
| GABR | 1.88 | 0.04 | 7.95 | 0.003 | 1.91 | 0.042 |
| | (1.04, 3.4) | | (2.03, 31.1) | | (1.02, 3.60) | |

*Hazard ratio comparing first and fourth quartiles (95% confidence interval)
†Adjusted for Framingham Risk Score, CrCl, and Log(hsCRP)

### Discussion

There are several key findings regarding the role of methylated derivatives of amino acids in this large, well-characterized patient population. First, the potential prognostic utility of ADMA in significantly obstructive CAD severity and related events in a large cohort of patients undergoing elective diagnostic cardiac catheterization was confirmed. More importantly, the inventors identified for the first time that in addition to ADMA, plasma levels of other products of arginine methylation such as SDMA and MMA are also strongly associated (inversely in the case of MMA, Table 2) with the presence of significantly obstructive CAD and incident risks for MACE over the ensuing 3 year period. It is also important to recognize that these associations remained robust even following adjustments for renal function as well as both traditional risk factors and hsCRP. Known as a “uremic toxin,” a potential confounding factor on plasma levels of ADMA and other methylarginine is the presence of renal insufficiency and underlying inflammation. Kielstein et al., Am J Kidney Dis., 46, 186-202 (2005). However, after multivariable adjustments incorporating CrCl and hsCRP, analytes of arginine methylation pathways alternative to ADMA (e.g. SDMA) and the integrated methylation index, ArgMI, still demonstrated independent prognostic value for cardiovascular events. The significant associations between both prevalent significantly obstructive CAD and incident major adverse events and (i) elevations in plasma levels of SDMA (in the absence of a NOS inhibitory effect); (ii) reductions in plasma levels of the potent NOS inhibitor MMA; and (iii) increased levels of ArgMI, a ratio that serves as an overall gauge for more extensive post translational methylation of arginine, suggests that the relationship between arginine methylation pathways and CAD initiation and progression extends well beyond direct NOS inhibition. The lack of prognostic value of plasma methyl-lysine levels argues against the contribution to long-term adverse cardiovascular risks (at least in a high-risk asymptomatic population) by other amino acid (non-arginine) methylation pathways. Taken together, the data demonstrate that not just the endogenous NOS inhibitor ADMA but other methylated arginine metabolites can collectively provide clinically important information relevant in the assessment of prevalent significantly obstructive CAD and long-term cardiovascular risks.

Despite the generally well-accepted prognostic role of ADMA in cardiovascular disease, there has been limited understanding regarding what specific pathophysiologic processes are at play. There has been a long-standing belief that the direct inhibition of NOS is the primary mechanism for the heightened cardiovascular risks associated with elevated ADMA levels. Indeed, animal model studies employing either infusion of ADMA or over-expression of DDAH-1 (major catabolic pathway for ADMA) have corroborated a potential role for arginine methylation derivatives and their catabolic pathways in the modulation of NO bioavailability, particularly under conditions where elevated ADMA are present. Jacobi et al., Circulation, 111, 1431-1438 (2005). Stuhlinger et al., Circulation, 108, 933-938 (2003). Tanaka et al., Circulation, 112, 1549-1556 (2005). Prior studies in humans have identified the association between SDMA and early renal compromise and the presence of coronary artery disease. Bode-Boger et al., J Am Soc Nephrol. 17, 1128-1134 (2006). Kielstein et al., Nephrol Dial Transplant, 21, 2446-2451 (2006). However, the present large prospective study substantially extends these observations by demonstrating that even in a population with normal renal function and following adjustments for traditional CAD risk factors, creatinine clearance, and markers of inflammation (hsCRP), plasma SDMA remains a significant predictor of both prevalent CAD risks and incident risks for MI or stroke, death, and MACE. Moreover, this is the first demonstration that combining SDMA and MMA with ADMA, provide added prognostic value. These findings are unexpected, as SDMA (a stereoisomer of ADMA) has been demonstrated to have no inhibitory effects on NOS. Closs et al., Nitric Oxide, 1, 65-73 (1997) However, it is clear that ADMA and SDMA are naturally occurring analogues of L-arginine, the substrate for NO synthesis by nitric oxide synthases (NOSs). Therefore, their structural similarities with L-arginine may suggest a direct inhibition of a common pathway via competition with L-arginine for transport into the cells through the cationic amino acid transporter. It is conceivable that direct competition may limit cellular entry of L-arginine, thereby accentuating intracellular L-arginine (substrate) deficiency leading to a decrease in NO production. Direct comparison with substrate estimates of arginine bioavailability (via GABR) indicates that this arginine methylation process may be particularly important in patients with more advanced CAD as the independent prognostic value of ArgMI were higher in secondary prevention cohort.

Perhaps one of the more remarkable findings in the present study is the inverse relationship between MMA levels and both prevalent significantly obstructive CAD and incident adverse cardiovascular events. This finding argues strongly for a rethinking of our understanding of arginine methylation in cardiovascular risk prediction. Among the methylarginines, MMA is the most potent NOS inhibitor and is widely employed as a pharmacological inhibitor of NOSs. Cardounel et al., J Biol Chem, 282, 879-887 (2007). While present at lower levels in plasma, the study clearly identifies the unexpected inverse relationship between plasma levels of this potent NOS inhibitor and CAD risks (Table 2). This study is also the first to examine plasma levels of MMA alone or in combinations with ADMA and SDMA in relationship to prevalent and incident CAD risks. Further studies are needed to validate these findings, but the present data clearly illustrates the complexity of arginine methylation metabolic pathways and the importance of understanding globally the “arginine/NO metabolome” rather than examining levels of single specific markers since each is involved in multiple interconnecting metabolic pathways. It is therefore conceivable that the increased risk associated with relatively low plasma level of MMA is a reflection of heightened post translational modification of proteins through di-methylation reactions and proteolysis, producing ADMA and SDMA in patients with cardiovascular diseases or those at risk of cardiovascular events. Thus, augmentation of arginine methylation pathways as indicated by the proposed arginine methylation index, ArgMI, may provide a more comprehensive index of cardiovascular risk. The present findings also argue that inflammation and oxidation pathways, which are known to enhance protein arginine residue post translational modification by methylation and subsequent proteolysis, may indeed be a dominant mechanism accounting for the established association between ADMA and both prevalent cardiovascular disease and incident adverse events.

Since lysine methylation pathways have no known interactions with the production of NO or its endogenous inhibitors, no relationship was expected between methyl-lysine levels and cardiovascular disease, and sought to examine levels of this alternative methylated amino acid as a “control” for generalized protein catabolic activity. The studies show free methyl-lysine levels demonstrate limited prognostic value regarding either prevalence of significantly obstructive CAD or the prediction of future cardiovascular events in the population examined. By simultaneously measuring both methylarginines and methyl-lysine levels, this “internal control” further validates the important contribution of arginine methylation pathways in the disease progression of long-term cardiovascular disease.

Other studies have shown that systemic levels of arginine alone are a poor prognostic indicator, and several studies have recently reported that GABR, an integrated index of arginine bioavailability, has improved prognostic utility in prediction of major adverse cardiac events in several populations, including studies of subjects with cardiogenic shock, subjects with sickle cell disease, and most recently, in stable cardiology patients. Multilogistic regression analyses with models incorporating both indices, ArgMI and GABR reveal that both remain significant independent predictors of incident MACE risk over the ensuing 3 year period, strongly supporting the contention that both arginine/NO metabolome related indices retain prognostic utility when evaluated within the same cohort. Moreover, further analyses showed that ArgMI and GABR appear to be sensitive to different aspects of cardiovascular risk. ArgMI showed a superior prognostic utility in secondary prevention subjects compared to primary prevention, suggesting protein arginine methylation, proteolysis and downstream interference of nitric oxide production are biochemical pathways with greater association to pathophysiological processes relevant to later stages of atherosclerotic plaque progression or vulnerable plaque development. In contrast, the GABR showed improved prognostic value among primary prevention subjects compared to secondary prevention, suggesting that processes linked to plaque initiation may have greater sensitivity to substrate (arginine) bioavailability for nitric oxide production. These analyses illustrate the complexity of different aspects of the NO/Arg metabolome and their potential contributions to different aspects of the pathogenesis of CAD along the spectrum of disease evolution including initiation, progression and acute complications.

### Conclusion

Plasma levels of arginine mono- and di-methylation are associated with the presence of cardiovascular disease and incident adverse events. Higher levels of both ADMA and SDMA and lower levels of MMA were predictors of prevalent disease, and elevated SDMA and ADMA both were predictive of long-term risks of major adverse cardiac events (heart attack, stroke or death), even following adjustments for traditional risk factors, hsCRP levels, renal function and indices of global arginine bioavailability. Based upon these findings, an integrated quantification of arginine methylation in the form of an “arginine methylation index” [ArgMI=(ADMA+SDMA)/MMA] provided the strongest independent risk prediction for incident major adverse cardiac events in stable patients undergoing elective cardiac evaluation, and significantly added to the prognostic utility of traditional risk factors. These results suggest that arginine methylation provides important contributions to disease progression beyond direct NOS inhibition.

### Example 2

### Cox Proportional Hazards Analysis for Prognostic Value of ArgMI (Arginine Methylation Index=[(ADMA+SDMA)/MMA] within Systolic and Diastolic Heart Failure Subjects (the ADEPT Clinical Trial)

TABLE 4

| Variable | HR (95% CI) | p-value | Endpoint |
|---|---|---|---|
| (ADMA + SDMA)/MMA | 1.34 (1.03-1.65) | 0.029 | 5 year Death |
| (ADMA + SDMA)/MMA | 1.30 (1.02-1.57) | 0.032 | 5 year Death/tx |
| (ADMA + SDMA)/MMA | 1.25 (1.01-1.48) | 0.044 | 5 year Death/ |
| | | | tx/HF hosp |
| Ln [(ADMA + | 1.39 (1.04-1.79) | 0.026 | 5 year Death |
| SDMA)/MMA] | | | |
| Ln [(ADMA + | 1.36 (1.05-1.71) | 0.022 | 5 year Death/tx |
| SDMA)/MMA] | | | |
| Ln [(ADMA + | 1.32 (1.04-1.63) | 0.022 | 5 year Death/ |
| SDMA)/MMA] | | | tx/HF hosp |

The results presented in table 4 show that within subjects with heart failure, an elevated level of ArgMI serves as a prognostic marker for incident risk for death, death or heart transplantation, or the composite endpoint of death, transplantation or need for unscheduled hospitalizations for heart failure.

Subsequent analyses of subgroups within this cohort showed similar results. Namely, among subjects with either non-ischemic cardiomyopathy or cardiomyopathy, ArgMI showed significant prognostic value (p<0.05) at prediction of all outcomes (incident risk for death, death or heart transplantation, or the composite endpoint of death, transplantation or need for unscheduled hospitalizations for heart failure).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of identifying a subject's risk of experiencing a complication of cardiovascular disease comprising:
determining the levels of dimethylarginine and N-monomethylarginine in a biological sample obtained from the subject using an analytic device;
comparing the levels of dimethylarginine and N-monomethylarginine to obtain an arginine methylation index;
comparing the arginine methylation index to one or more control values; and
characterizing the subject's risk of experiencing a complication of cardiovascular disease as higher if the arginine methylation index is higher than the one or more control values and lower if the arginine methylation index is lower than the one or more control values.

2. The method of claim 1, wherein the dimethylarginine is SDMA.

3. The method of claim 1, wherein the dimethylarginine is ADMA.

4. The method of claim 1, wherein the dimethylarginine is (SDMA+ADMA).

5. The method of claim 1, wherein the method comprises identifying a subject's risk of experiencing a complication of cardiovascular disease within the near term.

6. The method of claim 1, wherein the biological sample is blood serum, plasma, urine, or sputum.

7. The method of claim 1, wherein the complication is one or more complications selected from the group consisting of heart failure, non-fatal myocardial infarction, stroke, angina pectoris, transient ischemic attacks, aortic aneurysm, aortic dissection, peripheral artery disease, cardiomyopathy, abnormal cardiac catheterization, abnormal cardiac imaging, stent or graft revascularization, risk of experiencing an abnormal stress test, risk of experiencing abnormal myocardial perfusion, and death.

8. The method of claim 1, wherein the analytic device is an ultraviolet or mass spectrometer.

9. The method of claim 1, wherein the subject is experiencing chest pains and the complication is a myocardial infarction, reinfarction, acute coronary syndrome, unstable angina, or death within the near term.

10. A method of characterizing a subject's risk of having cardiovascular disease, comprising:
determining the levels of dimethylarginine and N-monomethylarginine in a biological sample obtained from the subject using an analytic device;
comparing the levels of dimethylarginine and N-monomethylarginine to obtain an arginine methylation index;
comparing the arginine methylation index to one or more control values, and
characterizing the subject's risk of having cardiovascular disease as higher if the arginine methylation index is higher than the one or more control values and lower if the arginine methylation index is lower than the one or more control values.

11. The method of claim 10, wherein the dimethylarginine is SDMA.

12. The method of claim 10, wherein the dimethylarginine is ADMA.

13. The method of claim 10, wherein the dimethylarginine is (SDMA+ADMA).

14. The method of claim 10, wherein the biological sample is blood serum, plasma, urine, or sputum.

15. The method of claim 10, wherein the analytic device is an ultraviolet/visible detector or mass spectrometer.

16. A method of characterizing a subject's risk of developing cardiovascular disease, comprising:
determining the levels of dimethylarginine and N-monomethylarginine in a biological sample obtained from the subject using an analytic device;
comparing the levels of dimethylarginine and N-monomethylarginine to obtain an arginine methylation index,
comparing the arginine methylation index to one or more control values, and
characterizing the subject's risk of developing cardiovascular disease as higher if the arginine methylation index is higher than the one or more control values and lower if the arginine methylation index is lower than the one or more control values.

17. The method of claim 16, wherein the dimethylarginine is SDMA.

18. The method of claim 16, wherein the dimethylarginine is ADMA.

19. The method of claim 16, wherein the dimethylarginine is (SDMA+ADMA).

20. The method of claim 16, wherein the biological sample is blood serum, plasma, urine, or sputum.

21. The method of claim 16, wherein the analytic device is an ultraviolet/visible detector or mass spectrometer.

22. A method of evaluating the efficacy of cardiovascular therapeutic intervention in a subject with cardiovascular disease, comprising:
determining the levels of dimethylarginine and N-monomethylarginine using an analytic device in a biological sample obtained from the subject during or after cardiovascular therapeutic intervention;
comparing the levels of dimethylarginine and N-monomethylarginine to obtain an arginine methylation index;
comparing the arginine methylation index to a predetermined value; and
determining the cardiovascular therapeutic intervention to be efficacious if the arginine methylation index is lower than the predetermined value.

23. The method of claim 22, wherein the dimethylarginine is ADMA.

24. The method of claim 22, wherein the dimethylarginine is SDMA.

25. The method of claim 22, wherein the dimethylarginine is (SDMA+ADMA).

26. The method of claim 22, wherein the cardiovascular therapeutic intervention is administration of a therapeutic agent.

27. The method of claim 22, wherein the cardiovascular therapeutic intervention is a life style change.

28. The method of claim 22, wherein the predetermined value is based on the arginine methylation index derived from a comparable biological sample taken from the subject prior to cardiovascular therapeutic intervention.

29. The method of claim 22, wherein the biological sample is blood serum, plasma, urine, or sputum.

30. The method of claim 22, wherein the analytic device is an ultraviolet/visible detector or mass spectrometer.
